(19) United States
(12) Patent Application Publication
(10) Pub. No.: US 2002/0189031 A1
(43) Pub. Date: Dec. 19, 2002

(54) Use of salts for improving the absorption qualities of anionic direct dyes

(76) Inventors: Manuela Javet, Marly (CH); Christel Dousse, Courtepin (CH)

Correspondence Address: Striker Striker & Stenby, 103 East Neck Road, Huntington, NY, 11743, US

(21) Appl. No.: 10/069,853

(22) PCT Filed: Mar. 29, 2001
(86) PCT No.: PCT/EP01/03605

(30) Foreign Application Priority Data
Jul. 5, 2000 (DE) 100 32 752.4

(51) Int. Cl. A61K 7/13

(52) U.S. Cl. 8/405; 8/407

(57) ABSTRACT

The object of the present invention is the use of physiologically compatible salts of inorganic or organic acids for improving the absorption behavior of anionic, direct dyes, a 2-step method for dyeing fibers, as well as a multi-component agent for the non-oxidative dyeing of fibers (particularly keratin fibers, such as human hair), the one component of which contains a dyeing agent based on anionic, direct dyes and the other component of which contains the physiological compatible salts.

The object of the present invention is the use of physiologically compatible salts of inorganic or organic acids for improving the absorption behavior of anionic, direct dyes, a 2-step method for dyeing fibers, as well as a multi-component agent for the non-oxidative dyeing of fibers (particularly keratin fibers, such as human hair), the one component of which contains a dyeing agent based on anionic, direct dyes and the other component of which contains the physiological compatible salts.

In conventional, commercial non-oxidative dyeing agents (so called tinting agents), nonionic and cationic dyes, for example, from the group of nitro dyes, azo dyes or anthraquinone dyes and triphenylmethane dyes, are preferably used. Such tints usually are sufficiently gentle to the hair and survive several washings of the hair.

However, under certain circumstances (for example, because of legislation), dyeing agents, based on anionic dyes, are used predominantly in some countries, although their dyeing properties are inferior. Since keratin, if it is charged at all, is charged negatively, there may be repulsion of the also negatively charged anionic dye. For this reason, only relatively weak, nonuniform dyeings, which are not very resistant to washing, are obtained with anionic dyes in the acidic region and practically no dyeings at all are obtained with these dyes in neutral and basic media. Admittedly, the absorption behavior can be improved by the addition of so-called carriers or penetration accelerators, such as benzyl alcohol or phenoxyethanol. At the same time, however, the already relatively strong coloration of the skin is clearly intensified even more by anionic dyes. In view of the aforementioned disadvantages, anionic dyes have gained a special importance at the present time only in Japan, since none of the otherwise customary, direct dyes are contained in the Japanese positive list for approved hair dyes.

An improvement in the absorption behavior of anionic dyes with, at the same time, an acceptable coloration of the skin and good equalization capabilities therefore represents a still unsolved problem. Pursuant to the invention, this objective is accomplished by the use of physiologically compatible salts of inorganic or organic acids.

In principle, the addition of inorganic or organic salts to nonoxidative hair dyeing agents admittedly is known from the state of the art (for example, the European Offenlegungsschrift 0 806 199, the German patent 196 40 792 or the German Offenlegungsschrift 19 42 3907. However, the use of these salts to improve the absorption behavior of anionic, direct dyes is completely new and was also not predictable on the basis of the state of the art.

The object of the present invention therefore is the use of one or more physiologically compatible salts of inorganic and/or organic acids for improving the absorption behavior of anionic, direct dyes on keratinic fibers, such as human hair.

As salts, especially the chlorides, bromides, sulfates, lactates, tartrates, citrates, malates, glycolates, glycerophosphates, pantothenates, phosphinates, glutamates, gluconates, phosphates, formates, sorbates, aspartates, orotates, oxalates and acetates of sodium potassium, magnesium, calcium, ammonium, aluminum or zinc come into consideration, the salts of calcium, magnesium, sodium and potassium and especially sodium chloride, potassium chloride, magnesium chloride, calcium chloride and calcium pantothenate, as well as their mixtures being preferred. The salts of potassium and calcium, especially potassium chloride, calcium chloride and calcium pantothenate as particularly preferred here.

The salts can be used individually as well as in the form of a mixture of several salts. The salts can be fabricated here in solid form, being mixed with water or the dyeing agent before use, as well as in the form of an aqueous preparation.

Based in the aqueous preparation or the dyeing agent, the total amount of salt used is about 0.01 to 10% by weight, preferably 0.1 to 5% by weight and especially 0.2 to 2.5% by weight.

A further object of the present application is a multi-step method for dyeing keratin fibers, especially human hair, for which the fibers initially are pre-treated with an aqueous preparation containing one or more physiologically compatible salts of an inorganic and/or organic acid, and subsequently dyed with a dyeing agent containing at least one anionic direct dye.

To begin with, an aqueous preparation, containing the physiologically compatible salts, is applied, for example, by spraying an aqueous salt solution, on the keratin fibers. Subsequently, the dyeing agent is applied on the fibers, which have been pre-treated with the salt. Depending on the depth of color desired, the dyeing agent is allowed to act for about 5 to 60 minutes and preferably 15 to 30 minutes at a temperature of about 20° to about 50° C. and especially of 30° to 40° C. Subsequently the hair is rinsed with water, optionally washed with a shampoo and dried.

A further object of the present application is a multi-component kit for carrying out the dyeing method described above. The kit contains a first component, which contains at least one physiologically compatible salt of an inorganic or organic acid, and a second component, which contains at least one anionic, direct dye.

The first component may be present in solid form or also in the form of an aqueous preparation and contains at least one physiologically compatible salt of an inorganic or organic acid, preferably a chloride, bromide, sulfate, lactate, tartrate, citrate, malate, glycolate, glycerophosphate, pantothenate, phosphinate, glutamate, gluconate, phosphate, formate, sorbate, aspartate, orotate, oxalate or acetate of sodium, potassium, magnesium, calcium, ammonium, aluminum or zinc, the salts of calcium and potassium, and especially sodium chloride, potassium chloride, magnesium chloride, calcium chloride and calcium pantothenate, as well as their mixtures, and optionally additives being preferred. If the first component is present in solid form, it must be mixed with water before use. The salts of potassium and calcium, especially potassium chloride, calcium chloride and calcium pantothenate, are particularly preferred.

The total amount of salt used in the aqueous preparation is about 0.01 by 10% by weight, preferably 0.1 by 5% by weight and particularly 0.2 to 2.5% by weight.

The dyeing agent may be present in the form of a solution, especially an aqueous or aqueous alcoholic solution, a cream, a gel, an aerosol foam or an emulsion, and represents a mixture of the components of the dye with the usual additives for such preparations.

The following, in particular, can be named as anionic dyes: disodium 6-hydroxy-5-[(4-sulfophenyl)azo]-2-naphthalenesulfonate (CI 15985; Food Yellow No. 3; FD&C Yellow No. 6), disodium 2,4-dinitro-1-naphthol-7-sulfonate (CI 10316; Acid Yellow No. 1; Food Yellow No. 1), 2-indane-1,3-dion-2-yl)quinoline-x,x-sulfonic acid (mixture of mono- and disulfonic acid) (CI 47005; D&C Yellow No. 10; Food Yellow No. 13; Acid Yellow No. 3), trisodium 5-hydroxy-1-(4-sulfophenyl)-4-[(4-sulfophenyl)-azo]-pyrazole-3-carboxylate (CI 19140; Food Yellow No. 4; Acid Yellow No. 23), 9-(2-carboxyphenyl)-6-hydroxy-3H-xanthene-3-one (CI 45350; Acid Yellow No. 73; D&C Yellow No. 8), sodium 5-[(2,4-dinitrophenyl)-amino]-2-phenylamino-benzenesulfonate (CI 10385; Acid Orange No. 3), monosodium 4-[(2,4-dihydroxyphenyl)azo]-benzenesulfonate (CI 14270; Acid Orange No. 6), sodium 4-[(2-hydroxynaphth-1-yl)azo]-benzenesulfonate (CI 15510; Acid Orange No. 7), sodium 4-[(2,4-dihydroxy-3-[(2,4-dimethylphenyl)azo]-phenyl)azo]-benzenesulfonate (CI 20170; Acid Orange No. 24), disodium 4-hydroxy-3-[(4-sulfonaphth-1-yl)azo]-1-naphthalene-sulfonate (CI 14720; Acid Red No. 14), trisodium 6-hydroxy-5-[(4-sulfonaphth-1-yl)azo]-2,4-naphthalene-disulfonate (CI 16255; Ponceau 4R; Acid Red No. 18), trisodium 3-hydroxy-4-[(4-sulfonaphth-1-yl)azo]-2,7-naphthalene-disulfonate (CI 16185; Acid Red No. 27), disodium 8-amino-1-hydroxy-2-(phenylazo)-3,6-naphthalene-disulfonate (CI 17200; Acid Red No. 33), disodium 5-(acetylamino)-4-hydroxy-3-[(2-methylphenyl)azo]-2,7-naphthalene-disulfonate (CI 18065; Acid Red No. 35), disodium 2-(3-hydroxy-2,4,5,7-tetraiodo-dibenzopyran-6-one-9-yl)-benzoate (CI 45430; Acid Red No. 51), N-[6-(diethylamino)-9-(2,4-disulfophenyl)-3H-xanthene-3-ylidene]-N-ethylethaneammoni-um hydroxide, internal sodium salt (CI 45100; Acid Red No. 52), disodium 8-[(4-(phenylazo)-phenyl)azo]-7-naphthol-1,3-disulfonate (CI 27290; Acid Red No. 73), disodium 2′,4′,5′,7′-tetrabromo-3′,6′-dihydroxyspiro[isobenzofuran-1(3H),9′-[9H]xanthene]-3-one (CI 45380; Acid Red No. 87), disodium 2′,4′,5′,7-tetrabromo-4,5,6,7-tetrachloro-3′,6′-dihydroxyspiro[isobenzofuran-1(3H), 9′[9H]xanthene]-3-one (CI 45410; Acid Red No. 92), disodium 3′,6′-dihydroxy-4′,5′-diiodospiro-[isobenzofuran-1 (3H), 9′(9H)-xanthene]-3-one (CI 45425; Acid Red No. 95), disodium (2-sulfophenyl)di[4-(ethyl-((4-sulfophenyl)methyl)amino)-phenyl]-carbenium, betaine (CI 42090; Acid Blue No. 9; FD&C Blue No. 1), disodium 1,4-bis[(2-sulfo-4-methyl-phenyl)amino]-9,10-anthraquinone (CI 61570; Acid Green No. 25), monosodium internal sodium salt of bis[4-(dimethylamino)phenyl]-(3,7-disulfo-2-hydroxynaphth-1-yl)-carbenium (CI 44090; Food Green No. 4; Acid Green No. 50), internal sodium salt of bis[4-(diethylamino)phenyl](2,4-disulfophenyl)-carbenium, (2:1) (CI 42045; Food Blue No. 3; Acid Blue No. 1), internal calcium salt of bis[4-(diethylamino)phenyl](5-hydroxy-2,4-disulfophenyl)carbenium (2:1) (CI 42051; Acid Blue No. 3), sodium 1-amino-4-(cyclohexylamino)-9,10-anthraquinone-2-sulfonate (CI 62045; Acid Blue No. 62), disodium 2-(1,3-dihydro-3-oxo-5-sulfo-2H-indole-2-ylidene)-2,3-dihydro-3-oxo-1H-indole-5-sulfonate (CI 73015; Acid Blue No. 74), internal monosodium salt of 9-(2-carboxyphenyl)-3-[(2-methylphenyl)-amino]-6-[(2-methyl-4-sulfophenyl)amino]xanthylium, (CI 45190; Acid Violet No. 9), sodium 1-hydroxy-4-[(4-methyl-2-sulfophenyl)amino]-9,10-anthraquinone (CI 60730; D&C Violet No. 2; Acid Violet No. 43), bis[3-nitro-4-[(4-phenylamino)-3-sulfo-phenyl-amino]-phenyl]-sulfone (CI 10410; Acid Brown No. 13), disodium 5-amino-4-hydroxy-6-[(4-nitrophenyl)azo]-3-(phenylazo)-2,7-naphthalene-disulfonate (CI 20470; Acid Black No. 1), chromium complex of 3-hydroxy-4-[(2-hydroxy-naphth-1-yl)azo]-7-nitro-1-naphthalene-sulfonic acid (3:2) (CI 15711; Acid Black No. 52), disodium 3-[(2,4-dimethyl-5-sulfophenyl)azo]-4-hydroxy-1-naphthalenesulfonate (CI 14700; Food Red No. 1; Ponceau SX; FD&C Red No. 4), tetrasodium 4-(acetylamino)-5-hydroxy-6-[(7-sulfo-4-[(4-sulfophenyl)azo]naphth-1-yl)azo]-1,7-naphthalenedisul-fonate (CI 28440; Food Black No. 1), disodium 5-[(2-methoxy-5-methyl-4-sulfophenyl)azo]-6-hydroxy-2-naphthalene-sulfonate (CI 16035; Food Red 17; Curry Red; FD&C Red No. 40) and sodium 3-hydroxy-4-(3-methyl-5-oxo-1-phenyl-4,5-dihydro-1H-pyrazole-4-ylazo)-naphthalene-1-sulfonate, chromium complex (Acid Red No. 195).

The direct dyes are used in the dyeing agent in a total amount of about 0.01 to 10% by weight and preferably 0.1 to 5% by weight.

Aside from the anionic dyes, the dyeing agent may, if necessary contain further customary and suitable synthetic or natural direct dyes.

Customary additives in solutions, creams, emulsions, aerosol foams or gels are, for example, solvents such as water, low molecular weight aliphatic alcohols, such as ethanol, n-propanol and isopropanol, glycol ethers or glycols such as glycerin and 1,2-dihydroxypropane, which are used alone or in combination with one another. Furthermore, the tinting agent may contain additives, which are well-known for cosmetic agents, such as wetting agents or emulsifiers, from the classes of anionic, cationic, amphoteric or nonionic surface-active substances, such as fatty alcohol sulfates, ethoxylated fatty alcohol sulfates, alkyl, sulfonates, alkylbenzenesulfonates, alkyltrimethylammonium salts, alkybetaines, ethoxylated fatty alcohols, ethoxylated nonylphenols, fatty acid alkanolamides, ethoxylated fatty acid esters, the anionic nonionic and amphoteric wetting agents and emulsifiers generally being preferred. Furthermore, thickeners, such as higher molecular weight fatty alcohols, starch or cellulose derivatives, perfumes, hair pre-treating agents, conditioners, hair swelling agents, preservatives, Vaseline, paraffin oil and fatty acids, as well as other care materials, such as cationic resins, lanolin derivates, cholesterol, pantothenic acid and betaine may be contained. The components mentioned are used in amounts customary for such purposes. For example, the wetting agents and emulsifiers are used in concentrations of about 0.5 to 30% by weight (based on the dyeing agent), the thickeners are used in an amount of about 0.1 to 25% by weight (based on the dyeing agent) and the care materials are used in a concentration of about 0.1 to 5.0% by weight (based on the dyeing agent).

In a particular preferred embodiment, the dyeing agent of the second component additionally contains at least one surface active compound from the group of anionic, amphoteric and nonionic wetting agents and surfactants, the fabrication as a nonionic, anionic or amphoteric cream being particularly preferred. Due to the addition of the surface active compounds, a particularly slight coloration of the skin is achieved.

The pH of the ready-for-use dyeing agent is about 1 to 9, and especially 2.5 to 8.5. If necessary, the desired pH can be achieved by the addition of organic or inorganic acids or alkalizing agents, such as phosphoric acid, hydrochloric acid, acetic acid, salicylic acid, malonic acid, gluconic acid lactone or α-hydroxycarboxylic acids, such as glycolic acid, lactic acid, tartaric acid, citric acid or malic acid or alkalizing agents such as alkanolamines, alkylamines, alkali hydroxides, ammonium hydroxide, alkali carbonates, ammonium carbonates or alkali phosphates.

The aforementioned physiologically compatible inorganic and/or organic salts enable a clear improvement to be achieved in the absorption behavior of anionic dyes on keratin fibers such as hair, wool or silk, as a result of more intensive and durable dyeings can be achieved, while at the same time, the coloration of the skin is less.

Although the salts are very suitable for improving the absorption behavior of anionic, direct dyes on keratin fibers, they can also be used very advantageously for dyeing other natural or synthetic fibers, such as cotton, viscose, nylon or cellulose acetate, with anionic direct dyes.

The following examples are intended to explain the object in greater detail without limiting it to these examples.

### EXAMPLES

### Examples 1.1 to 1.5

### Tinting Agent

| | | |
|---|---|---|
| X | g | direct dye(s) of Table 1 |
| 20.0 | g | ethanol |
| Y | g | salts of an inorganic/organic acid of Table 1 |
| to 100.00 | g | water, fully desalinated |

The salt is added to the tinting agent directly before use. The tinting agent (60 g) is applied to bleached hair and distributed uniformly with a brush. After a period of action of 30 minutes at 40° C., the hair is rinsed with lukewarm water and then dried.

The result of this dyeing treatment is summarized in Table 1.

TABLE 1
Dyeing Results

| | | | Tint After |
|---|---|---|---|
| Example | Direct Dye(s) | Salt | Dyeing |
| 1.1 | Acid Black No. 1: 0.2 g | potassium chloride: | green |
| | Acid Yellow No. 1: 0.2 g | 0.55 g | |
| 1.2 | Acid Red No. 14: 0.4 g | calcium chloride: | red |
| | | 0.30 g | |
| 1.3 | Acid Black No. 1: 0.2 g | potassium chloride: | blue |
| | | 0.80 g | |
| 1.4 | Acid Yellow No. 1: 0.2 | magnesium chloride: | yellow |
| | | 1.00 g | |
| 1.5 | Acid Orange No. 7: 0.2 g | Sodium chloride: | orange |
| | | 1.00 g | |

### Example 2

### Tinting Cream

| | | |
|---|---|---|
| 1.22 | g | Steareth-20 |
| 12.30 | g | cetyl stearyl alcohol |
| 10.0 | g | ethanol |
| 0.005 | g | Acid Yellow No. 1 |
| 0.13 | g | Acid Orange No. 7 |
| 0.05 | g | Acid Black No. 1 |
| 0.026 | g | Acid Violet No. 43 |
| 0.058 | g | Acid Red No. 18 |
| to 100.00 | g | water |

Bleached hair initially is pretreated with a spray solution, which contains 1% by weight of calcium chloride and 1% by weight of potassium chloride. After this pretreatment, 50 g of the above tinting cream are applied on the hair. After a period of action of 30 minutes at 40° C. the hair is rinsed out with water and dried. A brown coloration is obtained

### Example 3

### Tinting Cream

| | | |
|---|---|---|
| 3.00 | g | sodium lauryl ether sulfate (28% aqueous solution) |
| 12.30 | g | cetyl stearyl alcohol |
| 20.00 | g | ethanol |
| 0.0625 | g | Acid Yellow No. 1 |
| 0.0625 | g | Acid Orange No. 7 |
| 0.0625 | g | Acid Black No. 1 |
| 0.0625 | g | Acid Violet No. 43 |
| 0.1500 | g | Acid Red No. 14 |
| to 100.00 | g | water |

Bleached hair initially is pretreated with a spray solution, which contains 5% by weight of calcium pantothenate. At the end of this pretreatment, 50 g of the tinting cream above is applied to the hair. After a period of action of 30 minutes at 40° C., the hair is rinsed out with water and dried. A brown coloration is obtained.

### Example 4

### Tinting Cream

| | | |
|---|---|---|
| 3.00 | g | sodium lauryl ether sulfate (28% aqueous solution) |
| 12.30 | g | cetyl stearyl alcohol |
| 20.00 | g | ethanol |
| 0.3000 | g | Acid Orange No. 7 |
| 0.2000 | g | Acid Red No. 33 |
| 0.2000 | g | FD & C Red No. 40 |
| to 100.00 | g | water |

Bleached hair initially is pretreated with a spray solution, which contains 5% by weight of calcium pantothenate. At the end of this pretreatment, 50 g of the tinting cream above is applied to the hair. After a period of action of 30 minutes at 40° C., the hair is rinsed out with water and dried. An orange-red coloration is obtained.

### Example 5

### Tinting Cream

| | | |
|---|---|---|
| 3.00 | g | sodium lauryl ether sulfate (28% aqueous solution) |
| 12.30 | g | cetyl stearyl alcohol |
| 20.00 | g | ethanol |
| 0.0430 | g | Acid Orange No. 7 |
| 0.0350 | g | Acid Black No. 1 |
| 0.1140 | g | Acid Violet No. 43 |
| 0.0580 | g | Acid Red No. 18 |
| to 100.00 | g | water |

Bleached hair initially is pretreated with a spray solution, which contains 5% by weight of calcium pantothenate. At the end of this pretreatment, 50 g of the tinting cream above is applied to the hair. After a period of action of 30 minutes at 40° C., the hair is rinsed out with water and dried. A violet coloration is obtained.

### Example 6

### Comparison Experiment

The tinting agents of examples 1 to 5 are in each case used for coloring hair with an without a pretreatment in the manner described in the examples. Subsequently the intensity of the dyeings obtained is compared. The results are summarized in Table 2 below.

TABLE 2

| | | Measured Color Values | | | |
|---|---|---|---|---|---|
| Dyeing Agent/Dyeing Method | | L | a | b | |
| Example 1.1: | with KCl | 44.83 | −21.94 | +12.76 | (green) |
| | without KCl | 57.94 | −17.62 | +10.13 | (light green) |
| Example 1.2: | with CaCl₂ | 42.20 | +58.04 | +8.24 | (red) |
| | without CaCl₂ | 61.60 | +35.45 | +4.23 | (pink) |
| Example 1.3: | with KCl | 37.48 | −8.17 | −15.27 | (blue) |
| | without KCl | 53.70 | −9.76 | 8.97 | (light blue) |
| Example 1.4: | with MgCl₂ | 79.96 | −7.71 | +83.77 | (yellow) |
| | without MgCl₂ | 81.24 | −9.52 | +59.22 | (light yellow) |
| Example 1.5: | with NaCl | 59.70 | +44.04 | +60.80 | (orange) |
| | without NaCl | 67.54 | +32.44 | +49.87 | (light orange) |
| Example 2: | with CaCl₂/KCl | 31.72 | +8.27 | +10.03 | (brown) |
| | without CaCl₂/KCl | 54.06 | 15.49 | +22.51 | (light brown) |
| Example 3: | with calcium pantothenate | 34.00 | +8.53 | +8.32 | (brown) |
| | | 42.29 | +9.49 | +9.77 | (light brown) |
| Example 4: | with calcium pantothenate | 39.62 | +59.03 | +27.22 | (red) |
| | | 47.73 | +54.61 | +30.64 | (orange-red) |
| Example 5: | with calcium pantothenate | 32.01 | +16.73 | −7.18 | (violet) |
| | | 38.85 | +16.49 | −7.22 | (light violet) |

The comparison examples show, without doubt, that dyeings, which are clearly more color intensive, are obtained from a pretreatment with an inorganic or organic salt.

The L*a*b* calorimetric values were determined with a Chromameter II Minolta instrument. The L value represents the brightness (the lower the L value, the greater is the color intensity), while the “a” value is a measure of the red portion (the greater the “a” value, the greater is the red portion). The “b” value is a measure of the blue portion of the color, a more negative “b” value indicating a greater proportion of blue.

Unless stated otherwise, all percentages given in the present application are percentages by weight.

1. A use of physiologically compatible salts of inorganic and/or organic acids for improving the absorption behavior of anionic, direct dyes on keratinic fibers.

2. The use of claim 1, wherein the salt is selected from chlorides, bromides, sulfates, lactates, tartrates, citrates. malates, glycolates, glycerophosphates, pantothenates, phosphinates, glutamates, gluconates, phosphates, formates, sorbates, aspartates, orotates, oxalates and acetates of sodium, potassium, magnesium, calcium, ammonium, aluminum or zinc.

3. The use of claim 2 or 3, wherein the salt is selected from sodium chloride, potassium chloride, magnesium chloride, calcium chloride and calcium pantothenate or their mixtures.

4. The use of one of the claims 1 to 3, wherein the salt is used in an aqueous preparation or in a dyeing agent in an amount of 0.01 to 10% by weight.

5. A method for dyeing keratinic fibers for which the fibers initially are pretreated with an aqueous preparation containing at least one physiologically compatible salt of an organic or inorganic acid and subsequently dyed with a dyeing agent containing at least one anionic, direct dye.

6. The method of claim 5, wherein the dyeing agent is allowed to act on the hair from 5 to 60 minutes at a temperature of 20 to 50° C., depending upon the color intensity desired, and the hair is then rinsed with water, optionally washed with a shampoo and dried.

7. A multi-component kit for dyeing keratinic fibers, wherein the kit contains a first component containing at least one physiologically compatible salt and a second component containing at least one anionic, direct dye.

8. The multi-component kits of claim 7, wherein the first component contains at least one chloride bromide, sulfate, lactate, tartrate, citrate, malate, glycolate, glycerophosphate, pantothenate, phosphinate, glutamate, gluconate, phosphates, formates, sorbates, aspartates, orotates, oxalate and acetate of sodium potassium, magnesium, calcium, ammonium, aluminum or zinc as well as, optionally, additives.

9. The multi-component kit of claim 7 or 8, wherein the second component contains at least one anionic direct dye.

10. The multi-component kit of claims 7 to 9, wherein the second component additionally contains at least one surface active compound from the group of anionic, amphoteric and nonionic wetting agents and surfactants.
